(19) United States
(12) Patent Application Publication
(10) Pub. No.: US 2007/0202078 A1
(43) Pub. Date: Aug. 30, 2007

(54) Method Of Treating Viral Infections

(76) Inventor: Howard J. Smith, Victoria (AU)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/568,176

(22) PCT Filed: Aug. 3, 2004
(86) PCT No.: PCT/AU04/01031

§ 371 Date: Nov. 30, 2006

Related U.S. Application Data
(60) Provisional application No. 60/494,828, filed on Aug. 13, 2003.

(51) Int. Cl. A61K 38/21; A61K 31/7056

(52) U.S. Cl. 424/85.4; 514/43

(57) ABSTRACT

A method of pharmaceutical therapy includes co-administering: (A) interferon or derivative thereof with a low dose of B ribavirin ≦400 mg/day or ≦6 mg/kg/day), or related compound, where (B) provides a clinically effective blood level in portal circulation but less than clinically effective blood level in peripheral circulation, thereby providing a systemic effect of interferon throughout the body but a selective effect of ribavirin in the liver. The method also provides for co-administration of (A) with a high dose of (B) (preferably 400-800 mg/day) where (B) is administered as a slow-release formulation to provide a sustained virologic response and reduced side effects. The method also provides for co-adminstering (C) antioxidant or other membrane protective agent with both interferon and ribavirin such that the hepatoprotective activity of (C) complements the virucidal effect of interferon and ribavirin. (C) may be administered as a systemic or low-dose, slow-release, liver-selective formulation.

The present invention relates to a method of treating viral infections such as hepatitis C infections with the combination of ribavirin and Interferon.

### BACKGROUND

Hepatitis C is a chronic form of viral hepatitis caused by the hepatitis C virus (HCV). Being a viral infection, it is a systemic disease; however the principal site of both cell damage and viral replication is the liver. The virus is transmitted by blood transfusion and by various percutaneous routes including self-injection and contact by damaged skin or membranes with an infected source.

The clinical features of the illness include a phase of acute hepatitis followed in 50-70% of cases by chronic hepatitis. For most patients with chronic hepatitis C (80% or more), the disease is relatively benign with chronic fatigue lasting for twenty or more years. In up to 20% cirrhosis of the liver eventually develops, and there is an increased incidence of primary carcinoma of the liver.

The immediate cause of Impaired liver function is hepatocyte destruction, although this appears to involve an immune component rather than being immediately due to the cytopathic effects of the virus. Hepatitis C is more aggressive and more serious in patients who have concomitant impairment of the immune system, including infection with Human Immune Deficiency Virus (H.I.V.). The progress of the disease and its response to treatment may be monitored by serial measurements of HCV viral load together with the various biochemical tests of liver function including serum bilirubin, alanine aminotransferase (ALT) and other enzymes.

The principal treatment of hepatitis C is interferon, such as interferon alfa 2b and the newer product pegylated interferon. At present all interferons used in clinical practice are administered parenterally. However, there are several new forms of interferon in preclinical and clinical development that may be administered orally.

Interferon alfa 2b, for example, is a naturally occurring molecule produced and secreted by cells in response to various virus infections including HCV. The molecule binds to receptors on the cell membrane, which in turn induce subcellular responses that act to inhibit viral replication. Clinical trials have shown that when interferon alfa 2b is administered parenterally for up to 6 months, the response rate to this treatment (measured by major fall in HCV levels) approaches 25%.

Ribavirin is an orally-administered synthetic nucleoside that has no effect on HCV viral load or hepatic histology when given as monotherapy. However, coadministration of parenteral interferon alfa 2b and oral ribavirin from 400mg up to 1200mg per day to patients with hepatitis C lifts the response rate to over 60%. Therefore, the combination of interferon alfa 2b and ribavirin has been used for hepatitis C, and in particular, for those patients who are resistant to monotherapy with any form of interferon.

U.S. Pat. No. 6,172,046 describes the treatment of patients having viral infections such as chronic hepatitis C infection involving a combination therapy using ribavirin in an amount of 400 to 1200 mg/day and a therapeutically effective amount of interferon-alpha for a time period of from 20 up to 80 weeks.

However, it has been found that this combination therapy when continued over the necessary long periods of time is associated with serious side effects of which haemolytic anaemia is the most important. In fact clinical trials have reported decreases in haemoglobin concentration of ≧20 g/L, ≧30 g/L, and ≧40 g/L in 31, 27, and 21% of patients respectively over 48 weeks of treatment. Similar changes have been seen after 24 weeks treatment. In most cases the decrease in haemoglobin occurred early in the treatment period. Inevitably, anaemia of this degree causes fatigue and lethargy, thereby aggravating the same symptoms that are produced by the disease process.

It has been found that these serious side effects occur principally during the use of the combination therapy.

Accordingly, there is a need to provide an improved combination therapy for treating viral infections such as hepatitis C in patients which ameliorates the side effects throughout the duration of the combination therapy and which produces a sustained virologic response in more patients than was previously possible.

### SUMMARY

In a first aspect the present invention provides a method of treating viral infections in a patient which method comprises co-administering to said patient a therapeutically effective amount of interferon with a low dose of ribavirin.

Preferably the ribavirin is administered orally and at a dose delivery rate sufficient to provide a clinically effective blood level in the portal vein and less than required to provide a clinically effective blood level in the peripheral circulation to thereby provide a dose-delivery rate having a selective antiviral and interferon potentiating effect in the liver.

In a further preferred aspect the low-dose of ribavirin is administered in a slow-release formulation to provide a clinically effective blood level in the portal vein and less than required to provide a clinically effective blood level in the peripheral circulation.

Surprisingly, because ribavirin is itself metabolised and progressively cleared from the body by the liver, presentation of ribavirin at a low-dose and/or as a slow-release formulation for oral administration for the treatment of hepatitis C, can achieve clinically effective and stable blood levels of the drug in the liver and portal circulation, but lower and subclinical levels of ribavirin are achieved in the systemic circulation. Accordingly the method of the present invention provides a stable dose delivery rate with a clinically selective effect in the liver. In this way, the administration of interferon achieves a systemic antiviral effect, but the additive and potentiating antiviral effect of ribavirin is concentrated and retained within the liver so that the systemic side effects of ribavirin, principally anaemia and its sequelae are avoided or substantially minimised.

In a preferred embodiment the ribavirin dose is less than 400 mg/day, more preferably in the range of from 5 to 399 mg/day and even more preferably from 20 to 350 mg/day. The dose of ribavirin may be varied according to the body weight of the patient. Preferably the dose will be less than 6 mg/kg/day more preferably less than 5 mg/kg/day and most preferably in the range of from 1 to 5 mg/kg/day.

In a second aspect the present invention provides a method of treating viral infections in a patient which method comprises co-administering to said patient a therapeutically effective amount of interferon with ribavirin that is administered as a slow release formulation.

The dose of ribavirin used in this aspect of the invention is typically from 5 to 800 mg/day. Although relatively high doses in the range of from 400 to 800 mg/day may be used, doses of less than 400 mg/day or less than 6 mg/kg/day are preferred and even more preferably the dose is from 5 to 399 mg/day.

Higher doses of ribavirin of up to 1200 mg/day are currently used for the clinical management of hepatitis C, but are limited by the relatively high risk of systemic side effects of the drug. The present invention therefore provides that when higher doses of ribavirin (400-800 mg/day) are administered as a slow-release formulation, a better therapeutic effect with portal and hepatic concentrations higher than those conventionally achieved with systemic administration of the drug can be achieved. With a slow-release formulation it is expected that clinicians will now be more confident in coprescribing a higher dose of ribavirin (i.e. above the 350-400 mg/day limit) with systemic interferon to patients with the expectation of a more complete or faster therapeutic response.

In a preferred aspect the present invention provides a method of treating viral infections in a patient which method comprises co-administering to said patient a therapeutically effective amount of interferon with a low dose of ribavirin as a slow-release formulation.

In a third aspect the present invention provides the use of a therapeutically effective amount of interferon with a low dose of ribavirin in the preparation of a medicament to treat viral infections such as hepatitis C viral infections in a patient.

In a fourth aspect the present invention provides a kit for use in the treatment of viral infections comprising a therapeutically effective amount of interferon in combination with ribavirin as a slow-release formulation.

In a fifth aspect the present invention provides a pharmaceutical composition for the treatment of viral infections in a patient comprising a therapeutically effective amount of interferon together with a low dose of ribavirin.

In a sixth aspect the present invention provides a method of treating viral infections in a patient which method comprises co-administering to said patient a therapeutically effective amount of interferon with a low dose of ribavirin and an antioxidant or other membrane protective agent wherein said ribavirin and said antioxidant or said other membrane protective agent are administered in systemic doses or as a low-dose, slow-release formulation.

Hypoxia of the liver sufficient to impair liver function over and above the effects of any disease process is a common condition in most forms of liver disease. The principal perfusion of the liver is at low pressure and with less than arterial oxygen content through the portal venous circulation. When any disease process, including hepatitis C, causes swelling of the liver cells, there is an increased resistance to blood flow, so that both the flow of blood and the delivery of oxygen content to the liver fall to levels that impair liver cell function. The hypoxia causes production of oxygen free radicals that induce a sequence of cell membrane damage, cell swelling, and deterioration of cell function.

Accordingly it is expected that administration of various forms of antioxidants or other membrane protective agents in accordance with the method of the sixth aspect will protect the liver from the underlying disease processes by inhibiting the production of free radicals or their effect on cell membranes.

In a preferred aspect such antioxidants or other membrane protective agents include silybum marianum, s-adenosyl-L-methionine, coenzyme Q, vitamin A, vitamin C, Vitamin E, L-diltiazem, D-diltiazem and other agents.

### DETAILED DESCRIPTION

Throughout the description and claims of the specification, the word “comprise” and variations of the word, such as “comprising ” and “comprises”, is not intended to exclude other additives, components, integers or steps.

The present invention applies principally to the treatment of hepatitis C infection and to the combination use of interferon and ribavirin where it is therapeutically desirable to administer ribavirin in low doses to achieve liver-selective drug delivery and thereby reduce side effects while retaining efficacy.

However, the invention also applies to the treatment of any other form of viral infection in which the main tissue damage and the principle site of viral replication is in the liver.

Further, any form of interferon or any derivative thereof may be used in the treatment of the viral infections, including but not limited to interferon alfa or pegylated interferon alfa. Accordingly, the forms of interferon contemplated are those which have been previously shown to have efficacy against hepatitis C or other forms of viral hepatitis. However, the invention also contemplates the use of future forms of interferon including those which may be administered orally in the management of hepatitis.

The term “interferon-alfa” as used herein means the family of highly homologous species-specific proteins that inhibit viral replication and cellular proliferation and modulate immune response. Suitable interferon-alfas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon alpha-2c such as Berofor alpha 2 interferon available from Boehringer Ingelheim Pharmaceutical, Inc., Ridgefield, Conn., interferon alpha-n1, a purified blend of natural alfa interferons such as Sumiferon available from Sumitomo, Japan or as Wellferon interferon alpha-n1 (INS) available from the Glaxo-Wellcome Ltd., London, Great Britain, or a consensus alpha interferon such as those described in U.S. Pat. Nos. 4,897,471 and 4,695,623 and the specific product available from Amgen, Inc., Newbury Park, Calif., or interferon alfa-n3 a mixture of natural alfa interferons made by Interferon Sciences and available from the Purdue Frederick Co., Norwalk, Conn., under the Alferon Tradename or recombinant interferon alpha available from Frauenhoffer Institute, Germany or that is available from Green Cross, South Korea. The use of interferon alfa-2a or alfa 2b is preferred. Since interferon alfa 2b, among all interferons, has the broadest approval throughout the world for treating chronic hepatitis C infection, it is most preferred. The manufacture of interferon alfa 2b is described in U.S. Pat. No. 4,530,901.

The term “pegylated interferon-alfa” as used herein means polyethylene glycol modified conjugates of interferon-alfa, preferably pegylated interferon alfa-2a, pegylated interferon alfa-2b, or a pegylated consensus interferon, more preferably pegylated interferon alfa-2a and pegylated interferon alfa-2b.

At present most forms of interferon are administered parenterally, preferably by subcutaneous IV or IM injection. However, several new forms of interferon are currently in preclinical and clinical development that may be administered orally. Accordingly, the present invention contemplates the use of ribavirin with any form of interferon or any derivative thereof including the future oral forms of interferon or such derivatives.

In a preferred embodiment Interferon alfa 2b is administered parenterally in an amount of from 2 to 10 million IU per week on a weekly, thrice weekly (“TIW”), every other day (“QOD”) or daily basis.

In another preferred embodiment the interferon alfa administered systemically is the pegylated interferon alfa-2b and in an amount of 0.5 to 2.0 micrograms per kilogram of body weight per week on a weekly, TIW, QOD or daily basis. Alternatively, the interferon alfa administered is the pegylated interferon alfa-2a and in an amount of 20 to 250 micrograms per kilogram of body weight per week on a weekly, TIW, QOD or daily basis.

The invention of liver-selective drug delivery applies to drugs with short half-lives that are administered orally in low doses and in slow-release formulations. In this way, clinically effective concentrations of a drug will be achieved in the portal circulation and within the liver itself. However, clinically effective blood levels will not be achieved within the peripheral or systemic circulation because 1) a significant portion of the drug is removed by the liver during first-pass, and 2) the relatively large volume of the systemic circulation compared with the smaller volume portal circulation creates a dilution effect.

Ribavirin, a synthetic nucleoside analogue and is described in the Merck Index, compound No. 8199, Eleventh Edition, is actively absorbed from the gastrointestinal tract but undergoes extensive first-pass metabolism by the liver where it is degraded to triazole carboxamide and triazole carboxylic acid metabolites. Both ribavirin and its metabolites are further excreted by the kidney. Therefore, ribavirin is suitable for administration at low dose (less than 400 mg/day) or at a higher dose but as a slow-release formulation to achieve liver-selective delivery that retains both the drug and its therapeutic effect within the liver. Ribavirin is also well absorbed from the gastrointestinal tract. Therefore it can be administered as a slow-release formulation with the confidence that it will be continue to be absorbed into the portal venous circulation as the formulation disintegrates and continues to release the drug while it descends through the duodenum and ileum.

The term “ribavirin” as used herein includes ribavirin or any analogue thereof that is found to have virucidal activity. Preferably any analogue of ribavirin is administered orally as a therapeutic medicine to potentiate the antiviral effects of any form of interferon or any derivative thereof within the liver. More preferably the analogue is administered to a patient as a low-dose, slow-release formulation to deliver the drug in a liver-selective manner.

Analogues of ribavirin may include 5′-amino acid esters of ribavirin, any pharmaceutically acceptable salt of a ribavirin amino ester, or any ribavirin derivative.

Hypoxia of the liver sufficient to impair liver function over and above the effects of any disease process is a condition commonly associated with most forms of liver disease. Accordingly, the present invention contemplates the administration of various forms of antioxidants or other such membrane agents to patients with liver disease to protect the liver cells over and above the therapeutic endeavour being to treat the underlying condition with virucidal or other therapy.

That is, the present invention provides for a combination therapy of a therapeutically effective amount of any form of interferon or any derivative thereof in combination with ribavirin (or an analogue thereof administered as a slow-release formulation and an antioxidant or other membrane protective agent.

Preferably the antioxidant or other membrane protective agent is administered in systemic doses or as a liver-selective formulation, that is, as a low-dose, slow-release formulation so as protect the liver from the underlying disease processes.

Antioxidants used may include a silybum marianum, s-adenosyl-L-methionine, coenzyme Q, vitamin A, vitamin C, Vitamin E, L-diltiazem, D-diltiazem and other agents.

Formulation for Slow-Release

There are many techniques to effect slow release of an active pharmaceutical agent from an orally administered formulation. The present invention contemplates formulating a low dose of ribavirin as a slow-release formulation to produce liver-selectivity, and it is intended to cover any method of slow-release formulation. These methods may include techniques designed to delay the disintegration of a capsule, tablet, or other vehicle, techniques designed to delay the solubility of a capsule, tablet or other vehicle, and techniques in which an active agent may be bound to a polymer or other large molecule such that absorption can not take place until the substance has been released from the polymer or other large molecule. The means of achieving these different methods of slow-release are varied and include well-known older methods, such as layers of shellac coating, and more modern techniques using synthetic and cellulose polymers.

The dosage forms according to the present invention may be controlled-release dosage forms. The mechanism of release of these dosage forms can be controlled by diffusion and/or erosion. In a further preferred aspect the controlled-release formulation comprises at least one polymer-coated multiparticulates, polymer-coated tablets or minitablets, or hydrophilic matrix tablets.

A slow-release formulation of ribavirin may be designed to release the drug over a period of about 6 to about 24 hours following administration, thereby permitting once-a-day administration. In some embodiments, formulations releasing the drug over extended periods of time may have more than one timed-release component to affect time coverage.

Thus the method provides a dose-delivery rate of ribavirin with a clinically selective effect in the liver. In this way the additive and potentiating effects of ribavirin on the antiviral effects of interferon are achieved at lower than the commonly employed systemic doses of the drug. The use of lower doses of ribavirin (less than 400 mg/day or less than 6 mg/kg/day) together with the concentration of ribavirin within the liver and portal circulation produces a reduction or avoidance of the side effects such as anaemia and its sequelae that are commonly observed after administration of ribavirin in systemic doses.

The method of the present invention also provides for the use of higher doses of ribavirin (400-800 mg/day) administered as a slow-release formulation to achieve a better therapeutic effect with portal and hepatic concentrations higher than those conventionally achieved with systemic administration of the drug. in this way, when co-prescribed with interferon, a greater therapeutic effect and higher cure rate will be achieved than when the ribavirin is given systemically and at higher doses but not in a slow-release formulation. It is expected that the use of ribavirin as described above will achieve a greater tolerance, acceptance and compliance by patients of the combination therapy.

The principle of liver-selective delivery of drugs can be described mathematically in the following way.

Consider a drug administered by mouth as a slow-release formulation to achieve steady state release into the bowel with uptake into the portal venous circulation. The drug is then partly metabolised by the liver.

Let the volume of blood passing through the portal circulation in unit time=V_{P} litres.

Let the total volume of the systemic circulation=V_{S} litres.

Let the concentration of drug in the portal vein=C_{S} mg/litre.

Let the concentration of drug in the systemic circulation=Cₛ mg/litre.

Drug absorbed from the GI tract in unit time−D_{A} mg.

Drug metabolised by the liver in unit time=D_{M} mg

Drug not metabolised by the liver in unit time=D_{A}−D_{M} mg=D_{NM} mg

Let the metabolic clearance=M

This must range from 0 (no clearance) to 1.0 (total clearance).

Then Cₚ is deterMined by the amount of drug absorbed into the finite VP plus the concentration in the drug recirculated.
Cₚ =D_{A}/V_{P}+C_{S}
i.e.,
D_{A} V_{P} (C_{P}−C_{S}) equation. 1

Drug metabolised is a function of clearance rate, portal venous concentration and portal volume per unit time.
D_{M}=M ×C_{P}×V_{P} equation. 2

Systemic concentration of drug is determined by the volume of the systemic circulation and the amount of drug not metabolised
C_{S}=D_{NM}/V_{S}
i.e.,
D_{NM} C_{S}×V_{S} equation. 3
By definition,
D_{A}=D_{M} +D_{NM}

Substituting equations 1, 2, and 3,
V_{P} (Cₚ−C_{S})=M×Cₚ×Vₚ +C_{S}×V_{S}
and
Cₚ[V_{P}(1−M)] C_{S} (V_{S} +V_{P})
such that
C_{P}/C_{S}=(V_{S}+V_{P})/V_{P}(1 −M) equation 4

When a drug is both metabolised by the liver and excreted by the kidney, a further variable needs to be considered, namely the portion of drug within the systemic circulation that is excreted by the kidney in the same unit time.

Let the renal clearance=R. This must range from 0 (no clearance) to 1.0 (total clearance), but the range of values will usually be low, firstly because only 20-25%% of the systemic blood volume (or less during exercise) passes through the kidney in each circulatory transit of the body. Secondly, the rate of renal excretion even of hydrophilic drugs is slower than the rate of hepatic extraction as evidence by the longer half-lives of those compounds that are excreted from the body by the kidney.

The net effect of renal excretion is a progressive fall in the systemic concentration of any drug excreted from the body by the kidney.

Therefore, the systemic concentration (Cs) becomes Cs(1−R). We can now adjust equation 4 as follows:

$${C}_{P} / {C}_{S} = \frac{( {V}_{S} + {V}_{P} )}{{V}_{P} ( 1 - M ) ( 1 - R )} . \qquad equation 5$$

This relationship may be interpreted in the following way.

When a drug is administered continuously as a slow-release formulation, the drug achieves stable gradients of concentration throughout the portal and systemic circulations.

The rate or metabolism by the liver for lipophilic drugs is generally much faster than the rate of excretion by the kidney. The effect of hepatic metabolism together with the small volume of the portal circulation is the key variable contributing to liver-selective drug delivery when a drug is administered as a slow-release formulation, but any degree of renal excretion will serve to accentuate the concentration gradient between the portal; and systemic circulation.

When there is no metabolic clearance of a drug by the liver (M=0), and in the absence of renal excretion (R=0), the concentration gradient between portal and systemic vessels during steady state release of a drug from a slow-release formulation is a function of their relative volumes of the two circulations.
C_{P}/CS=(V_{S}+Vₚ)/V_{P}

With total hepatic clearance, M=1, and C_{P}/C_{S} tends towards infinity. If the rate of metabolism by the liver saturates, M will decline at higher dose levels. Therefore liver selectivity will be greater at lower dose levels, and be maximal when there is no effective saturation of metabolism.

Additional renal excretion (R>0) will accentuate liver-selective delivery by reducing the systemic concentration. However this effect is likely to be modest because of the slow rate of excretion by the kidney, and the fact that most blood will transit the body several times before reaching the kidney.

Portal venous flow does vary. Therefore C_{P}/C_{S} will be higher under low-flow conditions, for example in cirrhosis, but be low in high-flow situations such as when there is an abnormal shunting of blood perhaps through fistulae.

It is also important to note that in contrast to the therapeutic interaction of interferon and ribavirin, their kinetic handling is entirely independent. Therefore, liver-selective delivery of low-dose ribavirin is independent of the systemic kinetics of interferon. In this way, their therapeutic interaction is restricted to the liver.

The invention will now be described with reference to the following examples. It is understood that the examples are provided by way of illustration of the invention and that they are in no way limiting to the scope of the invention.

### EXAMPLE A

The following clinical protocol may be used to administer the combination therapy of the present invention.

Treatment Regime:

A patient suffering from chronic hepatitis C may receive parenterally 3 million IU of Intron A (interferon alfa-2b) once weekly in combination with 350 mg/day of an oral dosage ribavirin which is bound to a synthetic polymer to form a slow-release formulation. Any study may be in two phases, the first being at least 28 weeks in duration and compares the therapeutic effect of the liver-selective formulation of ribavirin with that of conventional doses given as systemic formulations. A second phase of the study may be conducted in a subset or subsets of patients with treatments lasting 68 weeks or more, to ensure that the therapeutic effect is sustained.

It is expected that the use of lower doses of ribavirin (less than 400 mg/day or less than 6 mg/kg/day) together with the concentration of ribavirin within the liver and portal circulation, will produce a substantial reduction in the side effects throughout the duration of the treatment and a sustained virologic response in the patient.

### EXAMPLE B

The following clinical protocol may be used to administer the combination therapy of the present invention.

Treatment Regime:

A patient suffering from chronic hepatitis C may receive parenterally 3 million IU of Intron A (interferon alfa-2b) once weekly in combination with 600 mg/day of an oral dosage ribavirin which is bound to a synthetic polymer to form a slow release formulation.

Any study will be two phases, the first being at least 28 weeks in duration and will compare the therapeutic effect of the liver-selective formulation of ribavirin with that of conventional doses given as systemic formulations. A second phase of the study will be required in a subset or subsets of patients with treatments lasting 48 weeks or more, to ensure that the therapeutic effect is sustained.

It is expected that the use of higher doses of ribavirin administered as a slow-release formulation will give rise to a more complete or faster therapeutic response with lower side effects than what is achieved with conventional or systemic doses.

It should be understood that the invention herein above is susceptible to variations, modifications and/or additions other than those specifically described and that the invention includes all such variations, modifications, and/or additions, which fall within the spirit and scope of the above description.

The discussion of documents, materials, articles and the like is included in this specification solely for the purpose of providing a context for the present invention. It is not suggested or represented that any or all of these matters formed part of the prior art base or were common general knowledge in the field relevant to the present invention before the priority date of each claim of this application.

1. A method of treating viral infections in a patient which method comprises co-administering to said patient a therapeutically effective amount of interferon and a low dose of ribavirin.

2. A method according to claim 1, wherein the ribavirin is administered orally and at a dose delivery rate sufficient to provide a clinically effective blood level in the portal vein and less than required to provide a clinically effective blood level in the peripheral circulation to thereby provide a dose-delivery rate having a selective antiviral and interferon potentiating effect in the liver.

3. A method according to claim 1, wherein the low-dose of ribavirin is administered in a slow-release formulation to provide a clinically effective blood level in the portal vein and less than required to provide a clinically effective blood level in the peripheral circulation.

4. The method according to claim 3, wherein the formulation of ribavirin is a controlled-release formulation.

5. The method according to claim 4, wherein the controlled-release formulation releases ribavirin by a mechanism chosen from diffusion and erosion.

6. The method according to claim 4, wherein the controlled-release formulation of ribavirin comprises at least one of polymer-coated multiparticulates, polymer-coated tablets, polymer-coated minitablets, and hydrophilic matrix tablets.

7. The method according to claim 1, wherein the ribavirin dose is less than 400 mg/day.

8. A method according to claim 7, wherein the ribavirin dose is in the range of from 5 to 399 mg/day.

9. A method according to claim 8, wherein the ribavirin dose is in the range of from 20 to 350 mg/day.

10. A method according to claim 1, wherein the ribavirin dose is varied according to the body weight of the patient.

11. A method according to claim 10, wherein the ribavirin dose is less than 6 mg/kg/day.

12. A method according to claim 11, wherein the ribavirin dose is less than 5 mg/kg/day.

13. A method according to claim 12, wherein the ribavirin dose is in the range of from 1 to 5 mg/kg/day.

14. The method according to claim 13, wherein the viral infection is hepatitis C.

15. The method according to claim 1, wherein the ribavirin is in the form of at least one of a ribavirin ester, salt, or analogue of ribavirin shown to be effective as an antiviral agent.

16. The method according to claim 15, wherein the interferon is interferon alfa or pegylated interferon alfa.

17. The method of claim 16, wherein the interferon is interferon alfa 2b.

18. The method according to claim 17, wherein the interferon is administered parenterally.

19. The method according to claim 18, wherein the interferon is administered by subcutaneous IV or IM injection.

20. The method according to claim 19, wherein the interferon is administered parenterally in an amount of from 2 to 10 million IU per week on a weekly, thrice weekly (“TIW”), every other day (“QOD”) or daily basis.

21. The method according to claim 16, wherein the pegylated interferon alfa is pegylated interferon alfa-2b and is administered systemically in an amount of 0.5 to 2.0 micrograms per kilogram of body weight per week on a weekly, TIW, QOD or daily basis.

22. The method according to claim 16, wherein the pegylated interferon alfa is pegylated interferon alfa-2a and is administered systemically in an amount of 20 to 250 micrograms per kilogram of body weight per week on a weekly, TIW, QOD or daily basis.

23. A method of treating viral infections in a patient which method comprises co-administering to said patient a therapeutically effective amount of interferon with ribavirin which is administered as a slow release formulation.

24. The method according to claim 23, wherein the dose of the ribavirin used is in the range of from 5 to 800 mg/day.

25. The method according to claim 24, wherein the ribavirin dose is in the range of from 400 to 800 mg/day.

26. The method according to claim 24, wherein the ribavirin dose is less than 400 mg/day.

27. The method according to claim 26, wherein the ribavirin dose is in the range of from 5 to 399 mg/day.

28. A method of treating viral infections in a patent which method comprises co-administering to said patient a therapeutically effective amount of interferon with a low dose of ribavirin as a slow release formulation.

29. A method according to claim 28 further comprising an antioxidant or other membrane protective agent which is administered in systemic doses.

30. A method according to claim 28 further comprising an antioxidant or other membrane protective agent which is administered as a low-dose, slow-release formulation.

31. A method according to claim 28, further comprising an antioxidant or other membrane protective agent which is co-formulated with the ribavirin as a low-dose, slow-release formulation.

32. A use of a therapeutically effective amount of interferon with a low dose of ribavirin and optionally an antioxidant or other membrane protective agent in the preparation of a medicament to treat viral infections in a patient.

33. A kit for use in the treatment of viral infections comprising a therapeutically effective amount of interferon in combination with ribavirin and optionally an antioxidant or other membrane protective agent as a slow-release formulation.

34. At kit according to claim 33 wherein the kit comprises unit doses of ribavirin providing a dose delivery rate sufficient to provide a clinically effective blood level in the portal vein and less than required to provide a clinically effective blood level in the peripheral circulation to thereby provide a dose-delivery rate having a selective antiviral and interferon potentiating effect in the liver.

35. A kit according to claim 33 wherein the low-dose of ribavirin is administered in a slow-release formulation to provide a clinically effective blood level in the portal vein and less than required to provide a clinically effective blood level in the peripheral circulation.

36. A kit according to claim 35 wherein the slow-release formulation of ribavirin comprises at least one of polymer-coated multiparticulates, polymer-coated tablets, polymer-coated minitablets, and hydrophilic matrix tablets.

37. A kit according to claim 35 wherein the unit dose of ribavirin is less than 400 mg/day.

38. A kit according to claim 35 wherein the unit dose of ribavirin is less than 6 mg/kg/day.

39. A kit according to claim 33 wherein the ribavirin is in the form of at least one of a ribavirin ester, salt or analogue or ribavirin shown to be effective as an antiviral agent.

40. A kit according to claim 33 wherein the interferon is in a form for parenteral administration.

41. A kit according to claim 33 comprising unit doses of interferon for providing an amount of from 2 to 10 million IU per week by thrice weekly (“TIW”), every other day (“QOD”) or daily administration.

42. A kit according to claim 33 wherein the interferon is interferon alfa or pegylated interferon alfa.

43. A pharmaceutical composition for the treatment of viral infections in a patient comprising a therapeutically effective amount of interferon together with a low dose of ribavirin and optionally an antioxidant or other membrane protective agent.
